(19) United States
(12) Patent Application Publication
(10) Pub. No.: US 2024/0359054 A1
(43) Pub. Date: Oct. 31, 2024

(54) GOLF SWING PRACTICE SYSTEM FOR TRAINING SELECTIVE MUSCLE ACTIVATION AND GOLF TRAINING METHOD USING THE SAME

(71) Applicant: Korea Advanced Institute of Science and Technology, Daejeon (KR)

(72) Inventors: Hyung-Soon PARK, Daejeon (KR); Hangil LEE, Daejeon (KR)

(73) Assignee: Korea Advanced Institute of Science and Technology, Daejeon (KR)

(21) Appl. No.: 18/766,884

(22) Filed: Jul. 9, 2024

Related U.S. Application Data
(63) Continuation of application No. PCT/KR2023/015746, filed on Oct. 12, 2023.

(30) Foreign Application Priority Data
Jan. 18, 2023 (KR) 10-2023-0007119

(51) Int. Cl. A63B 24/00; A63B 69/36; A63B 71/06

(52) U.S. Cl. CPC A63B 24/0003; A63B 69/3632; A63B 71/0622; A63B 2214/00; A63B 2220/803; A63B 2220/833; A63B 2220/836; A63B 2230/60

(57) ABSTRACT

A golf swing practice system is provided. The golf swing practice system includes one or more electromyography (EMG) sensors attached to a specific muscle point on a body of a user and a golf swing analysis device that is connected to the EMG sensor to enable data communication, wherein the golf swing analysis device may include a sensor signal collector configured to receive an EMG signal of the user from the EMG sensor, a data comparison analyzer configured to compare and analyze EMG data of the user and a professional golfer to be compared, and a user interface configured to receive information about a muscle that the user needs to train and display an analysis result of the data comparison analyzer.

### CROSS-REFERENCE TO RELATED APPLICATION

This application is a continuation of International Application No. PCT/KR2023/015746 filed on Oct. 12, 2023, which claims the benefit of Korean Patent Application No. 10-2023-0007119 filed on Jan. 18, 2023, in the Korean Intellectual Property Office, the entire disclosure of which is incorporated herein by reference for all purposes.

### BACKGROUND

### 1. Field of the Invention

One or more embodiments relate to a golf swing practice system and a golf training method using the same. More specifically, embodiments relate to a golf swing practice system for helping selective muscle activation of the body of a user and a golf training method using the same.

### 2. Description of the Related Art

Today, golf is a popular sport that many people enjoy. To become better at golf, people practice imitating swing forms of professional golfers or receive training from professional coaches. There are a variety of golf training systems in the market, but most of them require specialized tools to directly adjust the trajectory of a golf club, and although they may be useful in a training situation, they cannot be used during actual rounds.

Many studies have recently been conducted to improve golf swing ability. Conventional studies on improving golf swing ability provide coaching feedback on kinematic or kinetic data such as the position, speed, and force of the body or a golf club to focus on improving golf swing ability.

The kinematic or kinetic data is fundamentally expressed by a musculoskeletal system, and analyzing high-level movement control during a golf swing may provide deeper insight into important differences between amateur golfers and professional players.

### SUMMARY

Embodiments are to provide a golf swing practice system for suggesting direct and intuitive feedback for training for a user and a golf training method using the same, by identifying a current condition and ability of the user based on biomechanical data of the user measured during a golf swing and comparing the biomechanical data of the user with data of professional golfers.

In addition, embodiments are to provide a golf swing practice system for analyzing a difference by comparing a muscle activation of a user with a muscle activation of professional golfers and a golf training method using the same.

Furthermore, embodiments are to provide a golf swing practice system that may be used anywhere during training or golf rounds and a golf training method using the same.

According to an aspect, there is provided a golf swing practice system for selective muscle activation, the golf swing practice system including one or more electromyography (EMG) sensors attached to a specific muscle point on a body of a user and a golf swing analysis device that is connected to the EMG sensor to enable data communication, wherein the golf swing analysis device may include a sensor signal collector configured to receive an EMG signal of the user from the EMG sensor, a data comparison analyzer configured to compare and analyze EMG data of the user and a professional golfer to be compared, and a user interface configured to receive information about a muscle that the user needs to train and display an analysis result of the data comparison analyzer.

The sensor signal collector may be further configured to receive an inertial sensor signal from the user or an inertial measurement unit (IMU) attached to a golf club, wherein the data comparison analyzer may be configured to estimate a section of a golf swing of the user using the inertial sensor signal and compare EMG data of the user and the professional golfer according to the estimated section the golf swing.

The data comparison analyzer may be configured to select a professional golfer to be compared by considering one or more of a skill level and physiological information of the user.

The data comparison analyzer may be configured to compare and analyze one or more of a similarity in a muscle activation pattern and a difference in a muscle activation between the user and the professional golfer, for each section of a golf swing.

The user interface may be configured to provide feedback on use of a specific muscle to the user, according to the analysis result.

The user interface may be configured to represent a difference in a muscle activation between the user and the professional golfer in a number or a graph, for each section of a golf swing.

The user interface may be configured to highlight and display a muscle area with a greatest difference in the muscle activation compared to the professional golfer.

The user interface may be configured to provide a comment about a possibility of an injury to a muscle of the user, based on the muscle activation of the user.

According to another aspect, there is provided a golf training method using a golf swing practice system including one or more EMG sensors attached to a specific muscle point on a body of a user and a golf swing analysis device that is connected to the EMG sensor to enable data communication, the golf training method including selecting, by the user, one or more muscles that require training, through a user interface of the golf swing analysis device, receiving, by the golf swing analysis device, an EMG signal measured by the EMG sensor according to golf swing motion of the user, comparing and analyzing, by the golf swing analysis device, an EMG sensor signal of the user and an EMG sensor signal of a professional golfer to be compared, and displaying, by the user interface of the golf swing analysis device, a result of a comparative analysis to the user.

The comparing and analyzing of the EMG sensor signals may include comparing and analyzing muscle activation of the user and the professional golfer, for each section of a golf swing.

Additional aspects of embodiments will be set forth in part in the description which follows and, in part, will be apparent from the description, or may be learned by practice of the disclosure.

According to embodiments, a golf swing practice system and a golf training method using the same may help adjust and train a golf swing posture of a user based on a muscle activation analysis result of the user.

In addition, according to embodiments, a golf swing practice system and a golf training method using the same may fundamentally improve athletic ability of a user and at the same time, may provide important feedback to reduce risk of an injury during a golf swing.

In addition, according to embodiments, a golf swing practice system and a golf training method using the same may be used anywhere during training or golf rounds while providing real-time feedback.

In addition, according to embodiments, a golf swing practice system and a golf training method using the same may help reduce injuries that may occur due to overuse of certain muscles during a swing, and may be used not only in a training environment but also in actual golf rounds.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a block diagram illustrating a configuration of a golf swing practice system according to an embodiment;

FIG. 2 is a diagram illustrating an example of using the golf swing practice system of FIG. 1;

FIG. 3 is a block diagram illustrating a configuration of the data comparison analyzer of FIG. 1 in detail;

FIG. 4 is a diagram illustrating a similarity analysis of a muscle activity pattern performed by the data comparison analyzer of FIG. 1;

FIG. 5 is a diagram illustrating content displayed by a user interface of a golf swing analysis device, according to an embodiment;

FIG. 6 is a diagram illustrating other content displayed by a user interface of a golf swing analysis device, according to an embodiment;

FIG. 7 is a diagram illustrating additional content displayed by a user interface of a golf swing analysis device, according to an embodiment; and

FIG. 8 is a flowchart illustrating a golf training method according to an embodiment.

### DETAILED DESCRIPTION

Hereinbelow, embodiments of the disclosure will be described in detail with reference to the accompanying drawings so that the embodiments may be readily implemented by one of ordinary skill in the technical field to which the disclosure pertains. However, the present disclosure may be implemented in many different forms and is not limited to the embodiments described herein. Also, parts irrelevant to the description are omitted from the drawings for a clear description of the disclosure.

The terms used herein are for the purpose of describing particular embodiments only and are not to be limiting of the embodiments. The singular forms “a”, “an”, and “the” are intended to include the plural forms as well, unless the context clearly indicates otherwise.

It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof. In addition, the components described in the embodiments of the present disclosure are shown independently to illustrate different characteristic functions, and this does not indicate that each component includes separate hardware or one software component. That is, for ease of description, each component is arranged and described as each component, and at least two of each component may be combined to form one component, or one component may be divided into a plurality of components to perform a function. Integrated embodiments and separate embodiments of each component are also included in the scope of the present disclosure as long as the embodiments do not deviate from the essence of the present disclosure.

In addition, the following embodiments are provided for a clearer description to those with average knowledge in the art, and the shapes and sizes of elements in the drawings may be exaggerated for a clearer description.

Hereinafter, embodiments of the disclosure will be described in detail with reference to the accompanying drawings.

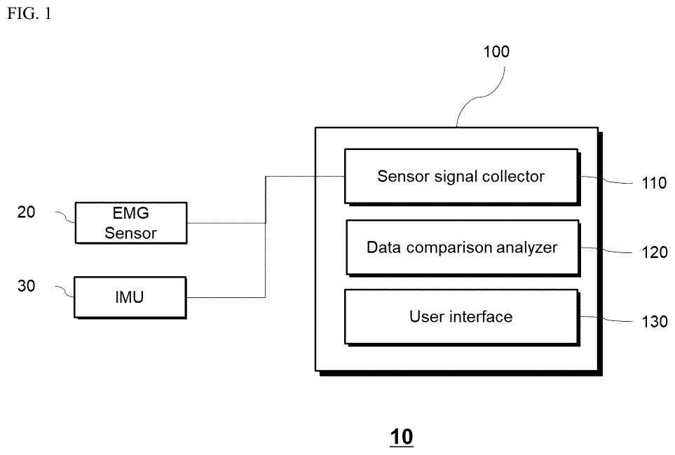

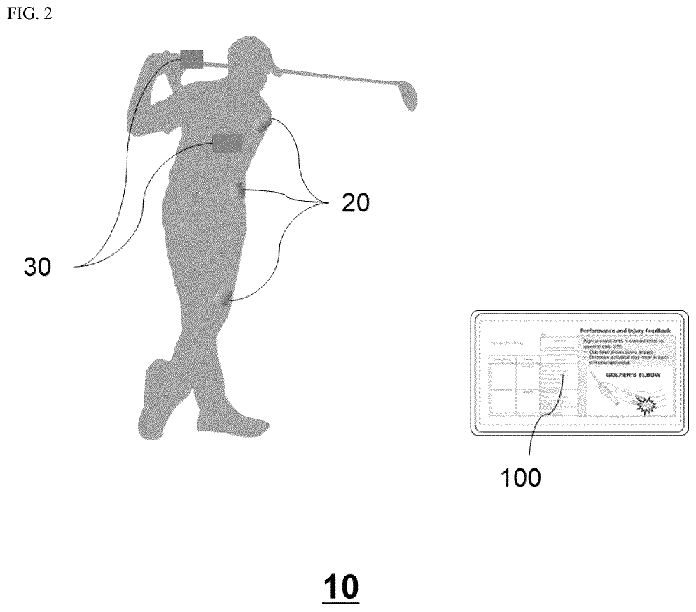

FIG. 1 is a block diagram illustrating a configuration of a golf swing practice system according to an embodiment, and FIG. 2 is a diagram illustrating an example of using the golf swing practice system of FIG. 1.

Referring to FIGS. 1 and 2, a golf swing practice system 10 according to an embodiment is designed to provide more effective golf swing training through selective muscle activation of a user performing a golf swing. The golf swing practice system 10 may include one or more electromyography (EMG) sensors 20 and a golf swing analysis device 100 that is communicatively connected to an EMG sensor 20. The golf swing practice system 10 may compare and analyze biomechanical data of the user to biomechanical data of a professional golfer in real time to identify a current condition and ability of the user and may then assist the user in golf training. The golf swing analysis device 100 may be provided in a portable form such as a tablet personal computer (PC), a computer, or a mobile device. The golf swing analysis device 100 may include a sensor signal collector 110, a data comparison analyzer 120, and a user interface 130.

The sensor signal collector 110 may be connected to the EMG sensor 20 to enable data communication. In an embodiment, the EMG sensor 20 may provide wireless communication and may transmit measured EMG data to the golf swing analysis device 100. In addition, depending on embodiments, the sensor signal collector 110 may receive data from an inertial measurement unit (IMU) 30 attached to the user or a golf club. By collecting data from the IMU 30, a swing motion of the user may be evaluated more accurately.

There may be provided a plurality of EMG sensors 20 attached to various locations on the body of the user to measure EMG signals at various points on the body of the user. For example, the plurality of EMG sensors 20 may output EMG signals related to left and right biceps femoris, left and right abdominal obliques, left and right pronator teres, left and right upper trapezius, and left and right vastus medialis of the user.

The plurality of EMG sensors 20 may be attached to muscles of the user to be trained and may measure an activation level and an activation pattern of each muscle for each section of a golf swing. The measured data may be used to evaluate whether a personalized training goal has been achieved through a comparative analysis with a database of professional golfers. For example, the user may over-activate or under-activate a certain muscle in a specific section of a golf swing, or a similarity may be low when compared to an activation pattern of a professional golfer. Here, after attaching a portable EMG sensor to relevant major muscles, more intuitive and fundamental training may be possible by inducing an activation of a targeted muscle during a swing.

The data comparison analyzer 120 may compare and analyze EMG data of a general user and a professional golfer. The data comparison analyzer 120 may include a biomechanical database including EMG data of professional golfers with various physical conditions. In addition, the biomechanical database of the professional golfers may be separately provided on an external server, and the golf swing analysis device 100 may access the external server through a network to receive biomechanical data of the professional golfers. The data comparison analyzer 120 may build a database by organizing biomechanical data of the general user into golf experience (e.g., a professional and an amateur), skill level (e.g., a handicap and years of experience), physiological information (e.g., height, weight, and age), and the like. The data comparison analyzer 120 may compare and analyze the biomechanical data of the general user and the professional golfers for each section of a golf swing, for example, for each section of a back swing, a downswing, an impact, and a follow swing. In this case, the data comparison analyzer 120 may consider one or more of the skill level and the physiological information (e.g., height, weight, and age) of the general user and may select a professional golfer suitable for the user as a comparison target. In addition, the data comparison analyzer 120 may estimate a section of a golf swing of the user using an inertial sensor signal from the IMU 30 and may compare and analyze the EMG data of the user and the professional golfer according to the estimated section of a golf swing.

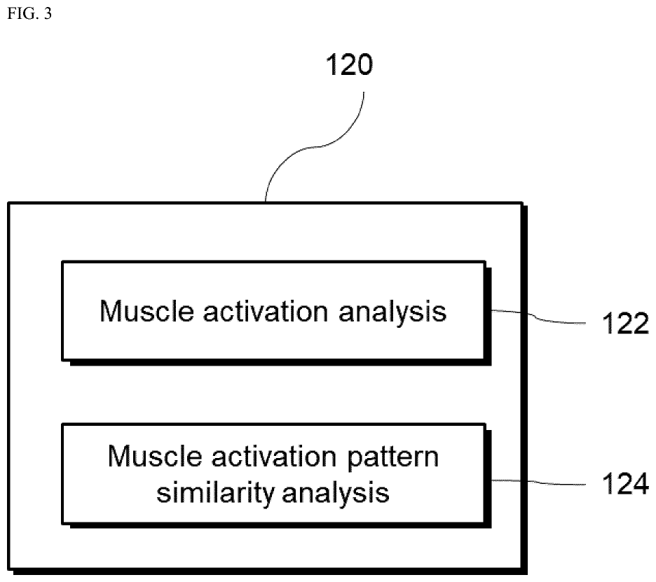

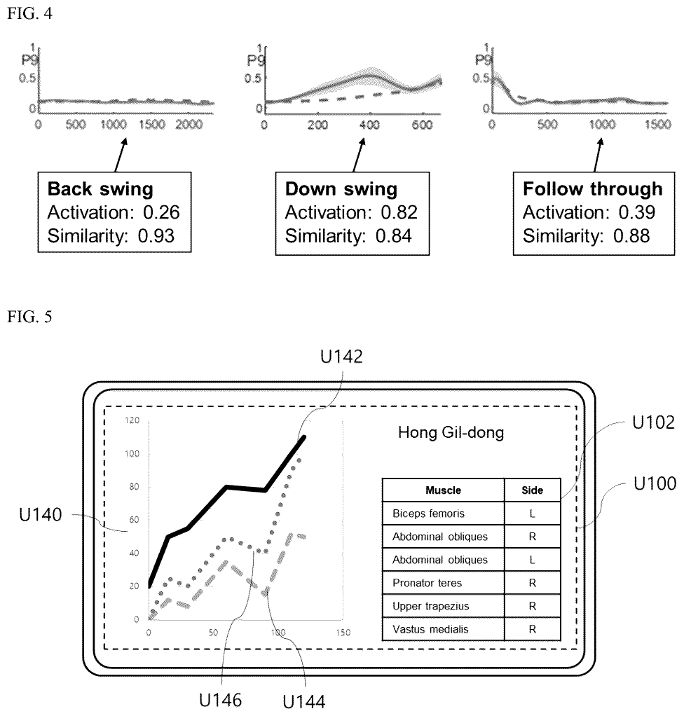

FIG. 3 is a block diagram illustrating a configuration of the data comparison analyzer of FIG. 1 in detail, and FIG. 4 is a diagram illustrating a similarity analysis of a muscle activity pattern performed by the data comparison analyzer of FIG. 1.

Referring to FIGS. 3 and 4, the data comparison analyzer 120 according to an embodiment may perform a muscle activation analysis 122 of a user and a similarity analysis 124 of a muscle activation pattern of a professional golfer to be compared with the user. Referring to FIG. 4, a solid line may represent a muscle activation signal of the professional golfer over time, and an area below the solid line may represent the total muscle activation of the professional golfer. In addition, a dashed line may represent a muscle activation signal of the general user over time, and an area below the dashed line may represent an average muscle activation of the general user. For example, in a section of a back swing motion, the muscle activation of the user is 0.26 and a similarity with the professional golfer is 0.93. In a section of a down swing, the muscle activation of the user is 0.82 and the similarity with the professional golfer is 0.84. In addition, in a section of a follow through, the muscle activation of the user is 0.39 and the similarity with the professional golfer is 0.88.

The user interface 130 may display a result of a comparative analysis between the general user and the professional golfer performed by the data comparison analyzer 120 to provide real-time feedback to the user. The user interface 130 may highlight and display important muscles for each section of a golf swing and may help users perform intuitive training by focusing on one section at a time. In addition, based on analysis data, the user interface 130 may suggest, to the user, muscles that need to be trained for each swing section and may also warn about overused muscles that are vulnerable to an injury during training. Hereinafter, the user interface 130 according to an embodiment is described in detail.

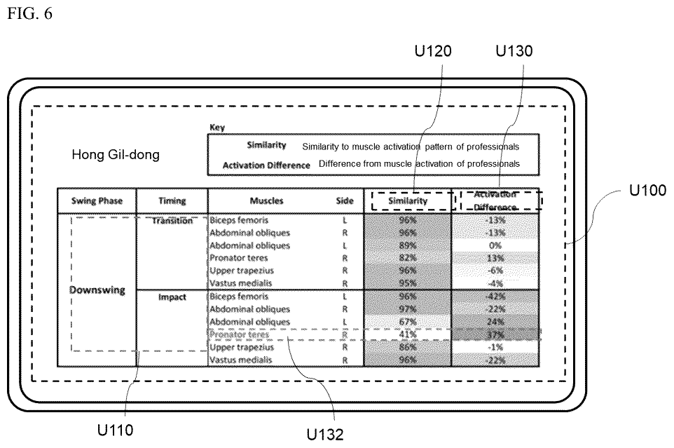

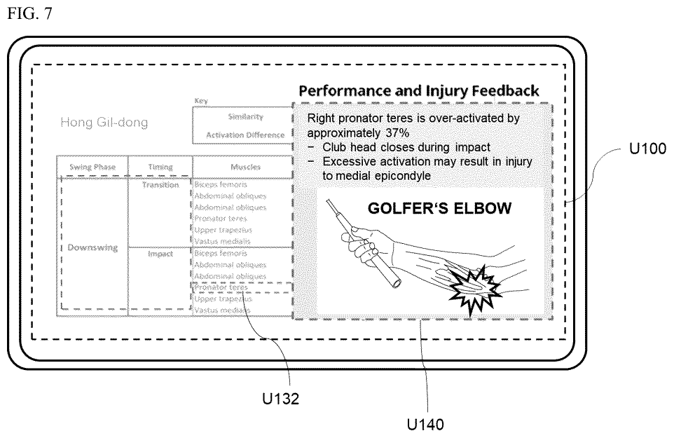

FIG. 5 is a diagram illustrating content displayed by a user interface of a golf swing analysis device, according to an embodiment, and FIG. 6 is a diagram illustrating other content displayed by a user interface of a golf swing analysis device, according to an embodiment. FIG. 7 is a diagram illustrating additional content displayed by a user interface of a golf swing analysis device, according to an embodiment.

Referring to FIG. 5, a user may input information about a muscle that needs training through a muscle selection inputter U102 of a user interface U100. In addition, when a golf swing is performed, the golf swing analysis device 100 may receive EMG sensor data from the EMG sensor 20, may analyze the EMG sensor data in real time, and may visually display data compared with a professional golfer suitable for the user, through a graph display U140. For example, regarding an activation of a specific muscle according to a swing section, the graph display U140 may display a muscle activation U142 of the professional golfer as a target activation and may also display a current muscle activation U146 of the user and a past muscle activation U144 of the user. Accordingly, the graph display U140 may help the user intuitively know how much the muscle activation of a current swing of the user differs from that of the professional golfer and how much the muscle activation has improved compared to the past.

Referring to FIG. 6, the user interface U100 according to an embodiment may include a swing section display U110 indicating a swing section and a timing in the swing section, a similarity display U120 indicating a similarity of an activation pattern with that of the professional golfer, according to a muscle measured in each swing section, and an activation difference display U130 indicating the difference between a muscle activation and that of the professional golfer. The similarity display U120 and the activation difference display U130 may numerically display the similarity of the activation pattern and the difference in the activation between each muscle of various points of the body of the user and that of the professional golfer. In addition, a display U132 may highlight and display a muscle with the greatest difference in the activation compared to that of the professional golfer, unlike other muscles.

Referring to FIG. 7, the user interface U100 according to an embodiment may provide a feedback comment U140 about a possibility of an injury to a muscle of the user, based on the muscle activation of the user. For example, the difference in the muscle activation compared to that of the professional golfer may be 37% in pronator teres, which indicates overactivation. This result may be provided as the feedback comment U140 with a reference diagram, indicating that a club head may be closed during an impact and an injury may be caused to medial epicondyle.

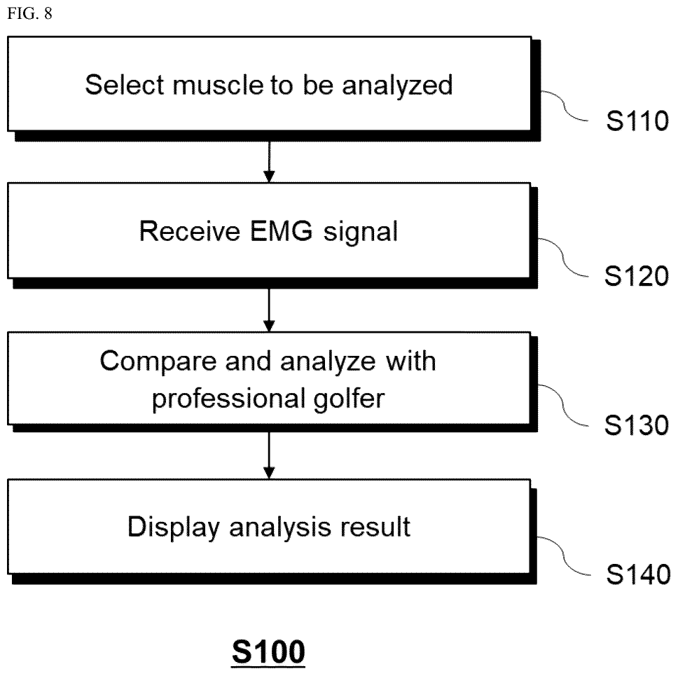

FIG. 8 is a flowchart illustrating a golf training method according to an embodiment.

Referring to FIG. 8, according to an embodiment, a golf training method S100 using a golf swing practice system including a golf swing analysis device may include operation S110 of selecting, by a user, one or more muscles that require training, through a user interface of the golf swing analysis device, operation S120 of receiving, by the golf swing analysis device, an EMG signal measured by the EMG sensor according to golf swing motion of the user, operation S130 of comparing and analyzing, by the golf swing analysis device, an EMG sensor signal of the user and an EMG sensor signal of a professional golfer to be compared, and operation S140 of displaying, by the user interface of the golf swing analysis device, a result of a comparative analysis to the user.

The operation S130 of the comparing and analyzing may include comparing and analyzing biomechanical data of the user and the professional golfer for each section of a golf swing to analyze the similarity and difference of a muscle activation for each major muscle between the user and the professional golfer, analyzed from the EMG sensor signals. The operation S140 of the displaying may include displaying, on the user interface in real time, a muscle activation of the professional golfer and a muscle activation of the user in a current swing in the form of a graph. In addition, the similarity and difference of the muscle activation between the user and the professional golfer may be displayed and specific feedback on a risk of an injury and a training method may be provided to the user for a muscle with a large difference.

The golf swing practice system and the golf training method using the same according to the above-described embodiments may propose personalized training content that helps adjust and train a golf swing posture of the user, based on a muscle activation analysis result of the user. In addition, after attaching portable EMG sensors to muscles that the user intends to train, the user may check, using a smartphone, what issues remain and what improvements have been made after performing a swing during a round of golf. Accordingly, the golf swing practice system and the golf training method may function as a portable personal coach.

Furthermore, an activation level and a pattern of specific muscles obtained through a pre-analysis may be corrected. The user may select important muscles for each swing section based on the analysis result and may proceed with training after attaching portable EMG sensors to the muscles. Therefore, a personalized training may be provided anywhere without any separate large device.

In addition, athletic ability of the user may be fundamentally improved and at the same time, important intuitive feedback may be provided anywhere to reduce risk of an injury during a golf swing. The golf swing practice system and the golf training method may also help reduce injuries that may occur due to overuse of specific muscles during a swing.

Various embodiments described herein may be implemented by hardware, middleware, microcode, software, and/or combinations thereof. For example, various embodiments may include one or more application-specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, and other electronic units designed to perform the functions described herein or a combination thereof.

In addition, for example, various embodiments may be encoded or embodied in a computer-readable medium including instructions. Instructions included or encoded in a computer-readable medium may cause a programmable processor or another processor to perform a method, for example, when the instructions are executed. Computer-readable media include both computer storage media and communication media including any medium that facilitates transfer of a computer program from one place to another. Storage media may be any available media that can be accessed by a computer. For example, such computer-readable media may include random-access memory (RAM), read-only memory (ROM), erasable programmable read-only memory (EEPROM), a compact disc read only memory (CD-ROM), or other optical disk storage media, magnetic disk storage media or other magnetic storage devices, or any other medium that may be used to carry or store the desired program code in the form of instructions or data structures accessible by a computer.

Although operations are shown in the drawings in a particular order, it should not be understood that these operations are performed in the particular order shown, or in a sequential order, or that all described operations need to be performed to achieve the desired results. In an arbitrary environment, multitasking and parallel processing may be advantageous. In addition, it should not be construed that the separation of various components of the aforementioned embodiments is required in all types of embodiments, and it should be understood that the described components are generally integrated as a single software product or packaged into a multiple-software product.

The present disclosure has been described with reference to the embodiments shown in the drawings, but this is merely an example, and it will be understood by one of ordinary skill in the art that various changes and equivalents may be made thereto. Accordingly, the true extent of technical protection of the present disclosure should be determined by the technical idea of the following claims.

What is claimed is:

1. A golf swing practice system for selective muscle activation, the golf swing practice system comprising:
one or more electromyography (EMG) sensors attached to a specific muscle point on a body of a user; and
a golf swing analysis device that is connected to the EMG sensor to enable data communication,
wherein the golf swing analysis device comprises:
a sensor signal collector configured to receive an EMG signal of the user from the EMG sensor;
a data comparison analyzer configured to compare and analyze EMG data of the user and a professional golfer to be compared; and
a user interface configured to receive information about a muscle that the user needs to train and display an analysis result of the data comparison analyzer.

2. The golf swing practice system of claim 1, wherein
the sensor signal collector is further configured to receive an inertial sensor signal from an inertial measurement unit (IMU) attached to the user or a golf club, and
the data comparison analyzer is configured to estimate a section of a golf swing of the user using the inertial sensor signal and compare EMG data of the user and the professional golfer according to the estimated section the golf swing.

3. The golf swing practice system of claim 1, wherein
the data comparison analyzer is configured to select a professional golfer to be compared by considering one or more of a skill level and physiological information of the user.

4. The golf swing practice system of claim 2, wherein
the data comparison analyzer is configured to compare and analyze one or more of a similarity in a muscle activation pattern and a difference in a muscle activation between the user and the professional golfer, for each section of a golf swing.

5. The golf swing practice system of claim 2, wherein
the user interface is configured to provide feedback on use of a specific muscle to the user, according to the analysis result.

6. The golf swing practice system of claim 1, wherein
the user interface is configured to represent a difference in a muscle activation between the user and the professional golfer in a number or a graph, for each section of a golf swing.

7. The golf swing practice system of claim 6, wherein
the user interface is configured to highlight and display a muscle area with a greatest difference in the muscle activation compared to the professional golfer.

8. The golf swing practice system of claim 6, wherein
the user interface is configured to provide a comment about a possibility of an injury to a muscle of the user, based on the muscle activation of the user.

9. A golf training method using a golf swing practice system comprising one or more electromyography (EMG) sensors attached to a specific muscle point on a body of a user and a golf swing analysis device that is connected to the EMG sensor to enable data communication, the golf training method comprising:
selecting, by the user, one or more muscles that require training, through a user interface of the golf swing analysis device;
receiving, by the golf swing analysis device, an EMG signal measured by the EMG sensor according to golf swing motion of the user;
comparing and analyzing, by the golf swing analysis device, an EMG sensor signal of the user and an EMG sensor signal of a professional golfer to be compared; and
displaying, by the user interface of the golf swing analysis device, a result of a comparative analysis to the user.

10. The golf training method of claim 9, wherein
the comparing and analyzing of the EMG sensor signals comprises comparing and analyzing muscle activation of the user and the professional golfer, for each section of a golf swing.
